(19) United States
(12) Patent Application Publication
(10) Pub. No.: US 2014/0031249 A1
(43) Pub. Date: Jan. 30, 2014

(54) MULTIPLEX MEASURE OF ISOTYPE ANTIGEN RESPONSE

(71) Applicant: SQI Diagnostics Systems Inc., Toronto (CA)

(72) Inventor: Peter Lea, Toronto (CA)

(73) Assignee: SQI Diagnostics Systems Inc., Toronto (CA)

(21) Appl. No.: 13/843,297

(22) Filed: Mar. 15, 2013

Related U.S. Application Data
(63) Continuation-in-part of application No. 11/632,746, filed on Mar. 26, 2008.

(30) Foreign Application Priority Data
Jul. 20, 2004 (CA) 2,475,240

(51) Int. Cl. G01N 33/543; G01N 33/564

(52) U.S. Cl. CPC G01N 33/54306; G01N 33/564
USPC 506/9

(57) ABSTRACT

Described are methods for simultaneous detection and quantifying multiple target analytes, including immunoglobulin isotypes and sub-classes, single and multiple protein antibodies within a test sample contained in a single reaction vessel. Such methods use reaction wells as on a multi-well plate, each single well comprising microarrays of calibration spots, each having a predetermined quantity of a target analyte; and capture spots, each having multiple agent antibodies, including isotypes and subclasses that specifically bind the target analytes. The captured analytes and the calibration spots are detected with fluorescently labeled antibodies specific for each different target analyte. Calibration spots generate calibration curves for quantitative determinations of different target analytes. Also described are methods for detecting and quantifying biomarkers, therapeutic proteins and patient derived antibodies; the use of secondary reagents to determine immunoglobulin classes Ig G, A, M, E and sub-classes including IgG1, IgG2, IgG3, IgG4 and IgA. The intensity of each fluorescent signal allows measurement of a specific immune response to a therapeutic protein and associated analytes; interrogates neutralizing effects of patient antibodies on therapeutic proteins, e.g., insulin therapy.

### CROSS-REFERENCE TO RELATED APPLICATIONS

This application is a continuation in part of co-pending U.S. Ser. No. 11/632,746, filed on Mar. 26, 2008, which is a national phase entry under 35 U.S.C. §371 of International Patent Application PCT/CA2005/001147, filed Jul. 20, 2005, published in English as International Patent Publication WO 2006/007726 on Jan. 26, 2006, which claims the benefit under 35 U.S.C. §119 and under Article 8 of the PCT to Canadian Patent Application No. 2,475,240, filed Jul. 20, 2004, the entire contents of each of which are incorporated herein by reference.

### TECHNICAL FIELD

The disclosure relates generally to biotechnology and more particularly to methods for the quantification of analytes and improved microarray methods for the detection and quantification of multiple analytes in a single sample. It also relates to the simultaneous quantification of isotype immunoglobulin classes Ig G, A, M, E, D and sub-classes including IgG1, IgG2, IgG3, IgG4, IgA1 and IgA2 within a single containment vessel. It further relates to microarray methods for the simultaneous detection and quantification of multiple analytes in a single sample. Analytical components may include subunits of therapeutic proteins, including antibody fragments or fusion partners, metabolic products, peptide components, formulation components, bio-similars or potential cross reacting entities.

### BACKGROUND

Current immunoassay methods detect one target per detection test cycle within a single reaction well. It is common for several antigenic substances or bio-markers to be associated with detection and diagnosis for any pathological or physiological disorder. To confirm the presence of multiple markers, each marker within a test sample often requires a separate and different immunoassay to confirm the presence of each target analyte to be detected. This often requires a multitude of tests and samples, increases delay in time to treatment, costs and possibility of analytical error. Current immunoassay methods do not detect antibodies that are of multiple isotypes and subclasses in the same test well/cycle.

Enzyme Linked Immunosorbent Assay (ELISA) was developed by Engvall et al., Immunochem. 8: 871 (1971) and further refined by Ljunggren et al. J. Immunol. Meth. 88: 104 (1987) and Kemeny et al., Immunol. Today 7: 67 (1986). ELISA and its applications are well known in the art.

A single ELISA functions to detect a single analyte or antibody using an enzyme-labelled antibody and a chromogenic substrate. To detect more than one analyte in a sample, a separate ELISA is performed to independently detect each analyte. For example, to detect two analytes, two separate ELISA plates or two sets of wells are needed, i.e. a plate or set of wells for each analyte. Prior art chromogenic-based ELISAs detect only one analyte at a time. This is a major limitation for detecting diseases with more than one marker or transgenic organisms which express more than one transgenic product.

Macri, J. N., et al., Ann Clin Biochem 29: 390-396 (1992) describe an indirect assay wherein antibodies (Reagent-1) are reacted first with the analyte and then second labelled anti-antibodies (Reagent-2) are reacted with the antibodies of Reagent 1. The result is a need for two separate washing steps which defeats the purpose of the direct assay.

US 2007141656 to Mapes et al. measures the ratio of self-antigen and auto-antibody by comparing to a bead set with monoclonal antibody specific for the self-antigen and a bead set with the self antigen. This method allows at least one analyte to react with a corresponding reactant, i.e. one analyte is a self-antigen and the reactants are auto-antibodies to the self antigen.

Another method for detecting multiple analytes is disclosed in US 2005118574 to Chandler et al which makes use of flow cytometric measurement to classify, in real time, sequential automated detection and interpretation of multiple biomolecules or DNA sequences, each biomolecule having to be separated and independently bound to a specific substrate particle, each particle being specific for separating and concentrating only a single species of analyte. This concentration of analyte is detected when the substrate particle is laser illuminated, as the particles flow, in single file, past the illuminating laser beam.

WO0113120 to Chandler and Chandler determines the concentration of several different analytes in a single sample. It is necessary only that there is a unique subpopulation of microparticles for each sample/analyte combination using the flow cytometer. These bead based systems' capability is limited to each microparticle, i.e., bead being suspended in a volume of test fluid that contains the analyte to be detected as a separate entity which needs to bind freely and specifically onto the surface of the test bead. Each bead effectively provides a requisite detection signal for only a specific analyte entity. Multiple, different entity binding events onto a single microparticle are not well distinguished or quantified using flow cytometry being restricted to multiple events of the same antibody isotype/subclass.

Simultaneous detection of more than one analyte, i.e. multiplex detection for simultaneous measurement of proteins has been described by Haab et al., “Protein microarrays for highly parallel detection and quantization of specific proteins and antibodies in complex solutions,” Genome Biology 2(2): 0004.1-0004.13, (2001), which is incorporated herein by reference. Mixtures of different antibodies and antigens were prepared and labelled with a red fluorescence dye and then mixed with a green fluorescence reference mixture containing the same antibodies and antigens. The observed variation between the red to green ratio was used to reflect the variation in the concentration of the corresponding binding partner in the mixes. This method is not suitable for quantitative results.

Mezzasoma et al. (Clinical Chemistry 48, 1, 121-130 (2002) published a microarray format method to detect analytes bound to the same capture spot in two separate assays, specifically different auto-antibodies reactive to the same antigen. The results revealed that when incubating the captured analytes with one reporter (for example that to detect immunoglobulin IgG), the corresponding analyte is detected. When incubating the captured analytes with the second reporter in an assay using a separate microarray solid-state substrate (for example to detect IgM), a second analyte (IgM) is detected.

WO0250537 to Damaj and Al-assaad discloses a method to detect up to three immobilized concomitant target antigens, bound to requisite antibodies first coated as a mixture onto a solid substrate. A wash step occurs before the first marker is detected. The presence of the first marker may be detected by adding a first specific substrate. The reaction well is read and a color change is detectable with light microscopy. Another wash step occurs before the second marker is detected. The presence of the second marker may be detected by adding a second substrate, specific for the second enzyme, to the reaction well. After sufficient incubation, the reaction well may be assayed for a color change. Similarly, a wash step may occur before the third marker is detected.

The presence of the third marker may be detected by adding a third substrate, specific for the third enzyme, to the reaction well. After sufficient incubation, the reaction well may be assayed for a color change. Although more than one analyte may be detected in a single reaction or test well, each reaction is processed on an individual basis.

WO2005017485 to Geister et al. describes a method to sequentially determine at least two different antigens in a single assay by two different enzymatic reactions of at least two enzyme labelled conjugates with two different chromogenic substrates for the enzymes in the assay (ELISA), which comprises (a) providing a first antibody specific for a first analyte and a second antibody specific for a second analyte immobilized on a solid support; (b) contacting the antibodies immobilized on the solid support with a liquid sample suspected of containing one or both of the antigens for a time sufficient for the antibodies to bind the antigens; (c) removing the solid support from the liquid sample and washing the solid support to remove unbound material; (d) contacting the solid support to a solution comprising a third antibody specific for the first antigen and a fourth antibody specific for the second antigen wherein the third antibody is conjugated to a first enzyme label and the fourth antibody is conjugated to a second enzyme label for a time sufficient for the third and fourth antibodies to bind the analytes bound by the first and second antibodies; (e) removing the solid support from the solution and washing the solid support to remove unbound antibodies; (f) adding a first chromogenic substrate for the first enzyme label wherein conversion of the first chromogenic substrate to a detectable color by the first enzyme label indicates that the sample contains the first analyte; (g) removing the first chromogenic substrate; and (h) adding a second chromogenic substrate for the second enzyme label wherein conversion of the second chromogenic substrate to a detectable color by the second enzyme label indicates that the sample contains the second analyte.

U.S. Pat. No. 7,022,479 to Wagner, entitled “Sensitive, multiplexed diagnostic assays for protein analysis”, is a method for detecting multiple different compounds in a sample, the method involving: (a) contacting the sample with a mixture of binding reagents, the binding reagents being nucleic acid-protein fusions, each having (i) a protein portion which is known to specifically bind to one of the compounds and (ii) a nucleic acid portion which includes a unique identification tag and which in one embodiment, encodes the protein; (b) allowing the protein portions of the binding reagents and the compounds to form complexes; (c) capturing the binding reagent-compound complexes; (d) amplifying the unique identification tags of the nucleic acid portions of the complex binding reagents; and (e) detecting the unique identification tag of each of the amplified nucleic acids, thereby detecting the corresponding compounds in the sample.

While methods for sequentially detecting and quantifying multiple analytes limited to capturing isotype and subclass for a single analyte are known, these methods require the use of separate assaying steps for each of the analytes of interest and as such, can be time consuming and costly, especially in the context of a clinical setting. A need exists for a method of sequentially detecting and quantifying multiple antibody isotypes and subclasses from a single sample using a single reaction vessel.

### SUMMARY OF THE DISCLOSURE

Provided is a fast and cost effective method for simultaneous detection and quantifying of multiple analytes in a test sample using a single reaction vessel. The method disclosed herein allows for the simultaneous detection of multiple analytes without the need for separate assays or reaction steps for each target analyte.

In one aspect, provided is a method for simultaneously detecting and quantifying two or more target analytes in a test sample comprising two or more target analytes:
a) providing a reaction vessel having a microarray printed thereon, the microarray comprising:
   i) a first calibration matrix comprising a plurality of the first calibration spots, each calibration spot comprising a predetermined amount of a first target analyte, the first target analyte being an antibody isotype or an antibody sub-class,
   ii) a second calibration matrix comprising a plurality of the second calibration spots, each calibration spot comprising a predetermined amount of a second target analyte, the second target analyte being an antibody isotype or antibody sub-class,
   iii) a first capture matrix comprising a plurality of first capture spots, each of the first capture spots comprising a predetermined amount of a first agent that selectively binds to the target analytes, and
   iv) a second capture matrix comprising a plurality of second capture spots, each of the second capture spots comprising a predetermined amount of a second agent that selectively binds to the target analytes;
b) adding a predetermined volume of the test sample to the microarray;
c) simultaneously applying at least two fluorescently labelled antibodies into the same well, each of the at least two fluorescently labelled antibodies being specific for one of the target analytes for selectively binding to one of the target analytes for individual identification and quantification of the target analytes, each of the fluorescently labelled antibodies comprising a different fluorescent dye having emission and excitation spectra which do not overlap with each other;
d) measuring signal intensity values for each of the analytes within each microarray spot;
e) generating calibration curves by fitting a curve to the measured signal intensity values for each analyte contained in each of the calibration spots versus a known concentration of the first target analyte and the second target analyte; and
f) determining the concentration for the first target analyte and the second target analytes using the generated calibration curves.

In a further embodiment, the reaction vessel is a well of a multi-well plate where the well has the microarray printed therein.

In a further embodiment, the test sample is a biological sample.

In another aspect, provided is a method for detecting and quantifying biomarkers diagnostic for immunogenicity testing of a therapeutic protein e.g. insulin, comprising the following steps:

a) providing an assay device having a microarray printed thereon, the microarray comprising:

i) a plurality of calibration matrices, each comprising a plurality of calibration spots, each calibration spot comprising a predetermined amount of a target analyte being an antibody selected from the group consisting of anti-insulin peptide human immunoglobulin classes Ig G, A, M, E and sub-classes including anti-insulin human immunoglobulin peptide sub-classes IgG1, IgG2, IgG3, IgG4 and IgA;

ii) an analyte capture matrix comprising a plurality of capture spots, each capture spot comprising a predetermined amount of an agent that selectively binds to the target analytes,

b) applying a predetermined volume of a serum sample to the assay device;

c) applying a plurality of different fluorescently labelled antibodies that selectively bind to human immunoglobulin classes Ig G, A, M, E, D and sub-classes including IgG1, IgG2, IgG3, IgG4 and IgA1, IgA2 respectively, the fluorescently labelled antibodies including a first fluorescently labelled anti-antibody that specifically binds to IgA antibodies, a second fluorescently labelled anti-antibody that selectively binds to IgG antibodies, a third fluorescently labelled anti-antibody that selectively binds to IgM antibodies, a fourth fluorescently labelled anti-antibody that selectively binds to IgE antibodies, a fifth fluorescently labelled anti-antibody that selectively binds to IgD antibodies, a sixth fluorescently labelled anti-antibody that selectively binds to sub-class IgG1, a seventh fluorescently labelled anti-antibody that selectively binds to sub-class IgG2, an eighth fluorescently labelled anti-antibody that selectively binds to sub-class IgG3, a ninth fluorescently labelled an anti-antibody that selectively binds to sub-class IgG4, wherein the first, second, third fourth, fifth, sixth, seventh and eighth fluorescently labelled antibodies each comprise a different fluorescent dye having emission and excitation spectra which do not overlap with each other;

d) measuring signal intensity values for each immunoglobulin contained within each spot forming the microarray printed in each well of the assay device;

e) generating calibration curves by fitting a curve to the measured signal intensity values for the each of the calibration spots versus the known concentration of the human IgA, IgG, IgE, IgM, IgD and subclass antibodies; and

f) determining the concentration for each of captured anti-insulin human isotype class and subclass immunoglobulins.

In another aspect, provided is a method for diagnosing multiplex immunogenicity, antibody and insulin factor immunogenicity in a subject, comprising:

a) measuring the concentration levels of class and subclass anti-insulin IgA, anti-insulin IgG, anti-insulin IgM, anti-insulin IgE; and

b) comparing the measured concentration levels of anti-insulin IgA, anti-insulin IgG, anti-insulin IgM, anti-insulin IgE, anti-insulin peptide immunoglobulins IgM with index normal levels of anti-insulin immunoglobulins and anti-insulin peptide immunoglobulins wherein measured concentrations levels which exceed index normal levels is diagnostic for insulin immunogenicity.

In an embodiment, the detection and quantification of predominantly anti-insulin IgM and anti-insulin peptide-IgM antibodies is diagnostic for an early stage of insulin immunogenicity.

In a further embodiment, the detection and quantification of anti-insulin IgA and anti-insulin peptide-IgA antibodies is diagnostic for a transitional stage of insulin immunogenicity.

In a further embodiment, the detection and quantification of anti-insulin IgG, anti-insulin peptide-IgG antibodies as well as their subclasses is diagnostic for a late stage of insulin immunogenicity.

In another aspect, provided is a method for monitoring reactions to immune stimuli by multiplex immuno testing, monitoring development of neutralizing antibodies, including, for example, specific insulin immunogenicity in a subject exposed to various insulin drug immune stimuli, using the method disclosed herein, a plurality of times in the course of treatments.

In another aspect, provided is a method for simultaneously detecting and quantifying two or more different target analytes in a test sample, comprising:

(a) providing a reaction vessel having a microarray printed thereon, the microarray comprising:

i) a calibration matrix comprising, for each of the target analytes, a plurality of calibration spots, each calibration spot comprising a predetermined known amount of a the target analyte in question,

iii) a capture matrix for each of the target analytes, comprising a plurality of capture spots, each capture spot comprising a predetermined amount of a binding agent that selectively binds to the target analyte in question,

b) applying to the microarray a predetermined volume of the test sample to the microarray;

c) applying to the microarray a fluorescently labelled antibody specific to each of the target analytes, wherein each fluorescently labelled antibody selectively binds to the target analyte in question, and wherein each of the fluorescently labelled antibodies comprises a different fluorescent dye having emission and excitation spectra which do not overlap with each other;

d) measuring signal intensity values for each spot within the microarray;

e) generating a calibration curve for each target analyte by fitting a curve to the measured signal intensity values of each of the calibration spots for the target analyte in question versus the known concentrations of the calibration spots for the target analyte; and

f) determining the concentration for each target analyte using the corresponding calibration curves for the target analyte.

According to yet another aspect, provided is a method for detecting and quantifying biomarkers diagnostic for rheumatoid arthritis, wherein the biomarkers comprise two or more target analytes in a serum sample, comprising:

a) providing an assay device having a microarray printed thereon, the microarray comprising:

i) a calibration matrix comprising, for each target analyte, a plurality of spots for each target analyte, each spot comprising a predetermined amount of the target analyte, and wherein the target analytes are a human IgA antibody, a human IgG antibody, and a human IgM antibody;

ii) a first analyte capture matrix comprising a plurality of spots, each spot comprising a predetermined amount of rheumatoid factor; and

iii) a second analyte capture matrix comprising a plurality of spots, each spot comprising a predetermined amount of cyclic citrullinated peptide;

b) applying a predetermined volume of the serum sample to the assay device;

c) applying a first fluorescently labelled antibody that selectively binds to IgA antibodies, a second fluorescently labelled antibody that selectively binds to IgG antibodies, and a third fluorescently labelled antibody that selectively binds to IgM antibodies to the assay device, wherein the first, second and third fluorescently labelled antibodies each comprise a different fluorescent dye having emission and excitation spectra which do not overlap with each other;

d) measuring signal intensity values for each spot within the assay device;

e) generating a calibration curve for each of the IgA, IgG and IgM antibodies, by fitting a curve to the measured signal intensity values of the calibration spots for the antibody in question versus the known concentration of the antibody; and

f) determining the concentration for each of captured rheumatoid factor-IgA, rheumatoid factor-IgG, rheumatoid factor-IgM, anti-cyclic citrullinated peptide-IgG, anti-cyclic citrullinated peptide-IgA, and/or anti-cyclic citrullinated peptide-IgM, in the first and second capture matrices, using the calibration curves for each of the IgA, IgG and IgM antibodies;

wherein the detection and quantification of rheumatoid factor antibodies, and

anti-cyclic citrullinated peptide antibodies is diagnostic for a stage of rheumatoid arthritis.

According to another aspect of the present invention, there is provided a method for simultaneously detecting and quantifying two or more different target analytes in a test sample, the method comprising:
(a) providing a reaction vessel having a microarray printed thereon, the microarray comprising: a capture matrix for each of the target analytes, comprising a plurality of capture spots, each capture spot comprising a predetermined amount of a binding agent that selectively binds to the target analytes in question, and
b) applying to the microarray a predetermined volume of the test sample to the microarray;
c) applying to the microarray a fluorescently labelled antibody specific to each of the target analytes, wherein each fluorescently labelled antibody selectively binds to the target analyte in question, and wherein each of the fluorescently labelled antibodies comprises a different fluorescent dye having emission and excitation spectra which do not overlap with each other;
d) measuring signal intensity values for each capture spot within the microarray;
e) determining the concentration for each target analyte with reference to an external standard.
According to an aspect of the present invention the method for simultaneously detecting and quantifying two or more different target analytes in a test sample is simultaneously carried out in multiple individual wells of a multiplex assay device, wherein each individual well detects multiple different antibody sub-classes and isotypes.

### BRIEF DESCRIPTION OF THE DRAWINGS

The patent or patent application file contains at least one drawing executed in color. Copies of this patent or patent application publication with color drawing(s) will be provided by the Office upon request and payment of the necessary fee.

Preferred embodiments hereof will now be described, by way of example, with reference to the accompanying drawings, in which:

FIG. 1 is a schematic illustration of the multiplex analyte detection method hereof

FIG. 2 is a schematic illustration of the multiplex analyte immunogenicity detection content per spot, in a single well, including IgG, IgA, IgM, IgE and subclasses for method of the disclosed invention, defined as Ig_plex.

FIG. 3 is a schematic illustration of a microarray printed on an assay device hereof

FIG. 4 illustrates typical 4 level multiplex microarray spots contained in a single well of an assay device hereof and

FIG. 5 is a plot to illustrate composite fluorescent intensity detection for six fluorophores at non-interfering wavelengths, at 457 nm (nanometers), 488 nm, 575 nm, 615 nm, 667 nm and 767 nm hereof.

FIG. 6 is a table showing the results 17 celiac patients and 7 normal patients to demonstrate overall agreement to single plex predicate

FIG. 7 is a schematic illustration of the multiplex analyte immunogenicity detection content per spot, in a single well, including IgA, IgG₁, and IgG₃.

FIG. 8 is a bar graph showing MFI from CEL serum on analyte singleplex (S) versus multiplexed (M).

FIG. 9 is a graph showing a plot of MFI versus sample dilution for Anti-Ig1-FITC, IgGA-APC and Anti-IgG3-RPE in a first multiplex well for CEL0058 on Analyte 3

FIG. 10 is a graph showing a plot of MFI versus sample dilution for Anti-Ig1-FITC, IgGA-APC and Anti-IgG3-RPE in a second multiplex well for CEL0167 on Analyte 3

FIG. 11 is an illustration depicting the effect of neutralizing antibodies

FIG. 12 is a graph showing a plot of raw fluorescence units versus inhibitor concentration for three samples showing IgG response.

FIG. 13 is a graph showing a plot of raw fluorescence units versus inhibitor concentration for three samples showing IgA response

FIG. 14 is a schematic illustration demonstrating multiplexed serum antibody specificity for serum IgG and IgA antibody for a drug molecule analog demonstrated in a single inhibition assay.

FIG. 15 is a graph showing a plot of raw fluorescence units versus acid concentration in terms of guanidine-HCl concentration.

### DETAILED DESCRIPTION

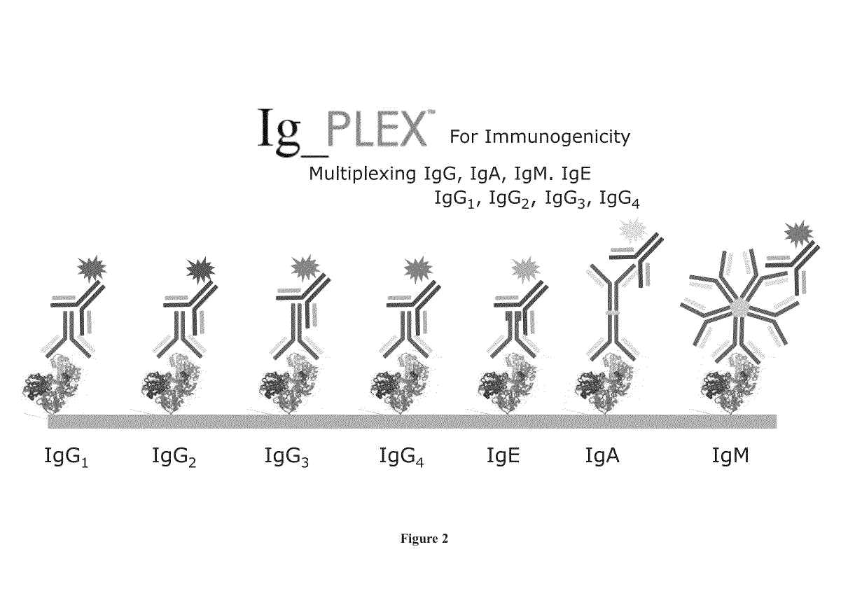

Provided is a method for the detection and quantification of multiple target analytes contained within each test spot or arrays of spots, of a test or test samples, within a single reaction well, per test cycle. The method disclosed herein provides for the simultaneous incubation of an assay device with two or more fluorescently labelled reporters in the same detection mixture as shown in FIG. 2. The method disclosed herein can detect a plurality of multiplexed analytes per test spot or capture spot, using a single reaction vessel instead of separate reaction vessels to detect each analyte. The terms “test spot” and “capture spot” can be used interchangeably for the purposes of the present specification.

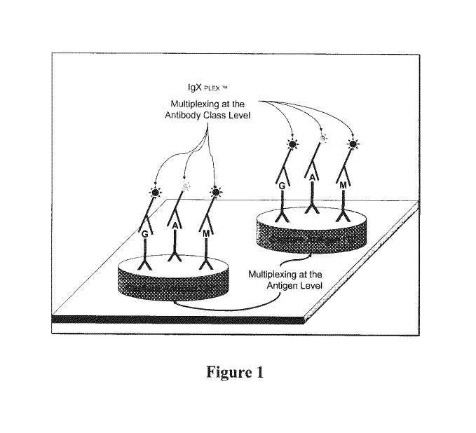

FIG. 1 illustrates the capturing of six different antibodies that selectively bind to two different antigens. The six different antibodies fall into three different antibody classes. In this example, an IgG is included that specifically binds to antigen A while a separate IgG is included that specifically binds to antigen B. Similarly, an IgA is included that specifically binds to antigen A while a separate IgA is included that specifically binds to antigen B. Finally, an IgM is included that specifically binds to antigen A while a separate IgM is included that specifically binds to antigen B. In such embodiments, only one calibration matrix may be required for each of the three different classes of immunoglobulins.

The methods disclosed herein can be used to detect and quantify biomarkers diagnostic for rheumatoid arthritis. In one embodiment, the method comprises the provision of an assay device having a microarray printed thereon. The microarray may comprise: i) a calibration matrix comprising plurality of spots, each spot comprising a predetermined amount of one of: a human IgA antibody, a human IgG antibody, and a human IgM antibody; ii) a first analyte capture matrix comprising a plurality of spots comprising a predetermined amount of rheumatoid factor; and iii) a second analyte capture matrix comprising a plurality of spots comprising a predetermined amount of cyclic citrullinated peptide. A predetermined volume of a biological sample, preferably a serum sample, is applied to the assay device. A cocktail comprising a first fluorescently labelled reporter compound that selectively binds to IgA antibodies, a second fluorescently labelled reporter compound that selectively binds to IgG antibodies, and a third fluorescently labelled reporter compound that selectively binds to IgM antibodies is then applied to the assay device. The first, second and third fluorescently labelled antibodies are chosen such that each of the antibodies comprise a different fluorescent dye having emission and excitation spectra which do not overlap with each other. A signal intensity value for each spot within the assay device is then measured using a single or multi-channel detector as discussed above. Using the measured signal intensity values, calibration curves are then generated by fitting a curve to the measured signal intensity values for the each of the calibration spots versus the known concentration of the human IgA, IgG and IgM antibodies. The concentration for each of captured rheumatoid factor-IgA, rheumatoid factor-IgG, rheumatoid factor-IgM, anti-cyclic citrullinated peptide-IgG, anti-cyclic citrullinated peptide-IgA, and/or anti-cyclic citrullinated peptide-IgM is the determined using the calibration curves.

In certain embodiments, the method can be used to diagnose or monitor the progress of autoimmune diseases. For example, in the case of rheumatoid arthritis, the detection and quantification of predominantly rheumatoid factor-IgM and anti-cyclic citrullinated peptide-IgM antibodies is diagnostic for an early stage of rheumatoid arthritis whereas the detection and quantification of rheumatoid factor-IgA and anti-cyclic citrullinated peptide-IgA antibodies is diagnostic for a transitional stage of disease progression and the detection and quantification of rheumatoid factor-IgG and anti-cyclic citrullinated peptide-IgG antibodies is diagnostic for a late stage of disease progression. In other embodiments, the method disclosed herein can be used to monitoring the progress of treatment in a subject suffering from rheumatoid arthritis. For example, the concentration levels of rheumatoid factor-IgA, rheumatoid factor-IgG, rheumatoid factor-IgM and at least one of anti-cyclic citrullinated peptide-IgG, anti-cyclic citrullinated peptide-IgA, and anti-cyclic citrullinated peptide-IgM can be measured a plurality of times during the treatment.

When the target analytes of interest are different classes of human antibodies e.g. hIgG, hIgA, hIgM, hIgE and their respective subclasses are directed to the same antigen (i.e. the Fc region of hIgG), the detection and quantification of each of the target antibodies requires separate assays when conventional methods are employed. With conventional methods, one assay is performed to detect and quantify the amount of hIgG present in a test sample. A second assay must be performed to detect and quantify the amount of hIgM and more assays must be performed to detect and quantify the presence of isoform classes and subclasses. In contrast, the method hereof eliminates the need for multiple detection steps thus reducing costs and time. Using the method hereof, target hIgG, hIgA, hIgM, hIgD and hIgE molecules contained in a test sample can be bound to a single capture spot in an assay device. In the disclosed method, the different classes of antibodies and antibody sub-classes can be detected in a single test by using a cocktail of fluorescently labelled antibodies directed to each of the isoform class hIgG, hIgM, hIgA, hIgE and respective subclass targets. As the antibodies are labelled with different optically excited and emitted fluorescent probes, each of the targets bound to a single capture spot can be detected and quantified using an appropriate calibrator. The use of multi-channel detectors allows for substantially simultaneous detection of multiplex analytes in a single assay. The spot morphology and density of capture molecules is optimized so as to mitigate for steric hindrance. As shown in FIG. 2, the target analytes are IgG, IgA, IgM, IgE, IgG 1, IgG2, IgG3, and IgG4. Capture spots include an antigen that binds to these antibody isotypes and subclasses. Once the target analyte antibodies bind to the capture spots, fluorescently labeled anti-IgG, anti-IgA, anti-IgM, anti-IgE, anti-IgG1, anti-IgG2, anti-IgG3, and anti-IgG4 bind to the respective target analyte antibodies so that the amount of bound IgG, IgA, IgM. IgE, IgG1, IgG2, IgG3, and IgG4 can be detected.

The methods employ assay devices useful for conducting immunoassays. The assay devices may be microarrays in 2 or 3-dimensional planar array format.

In one embodiment, the method may employ the use of a multi-well plate and wherein each well has a microarray printed therein. A single well is used as a reaction vessel for assaying the desired plurality of target analytes for each test sample.

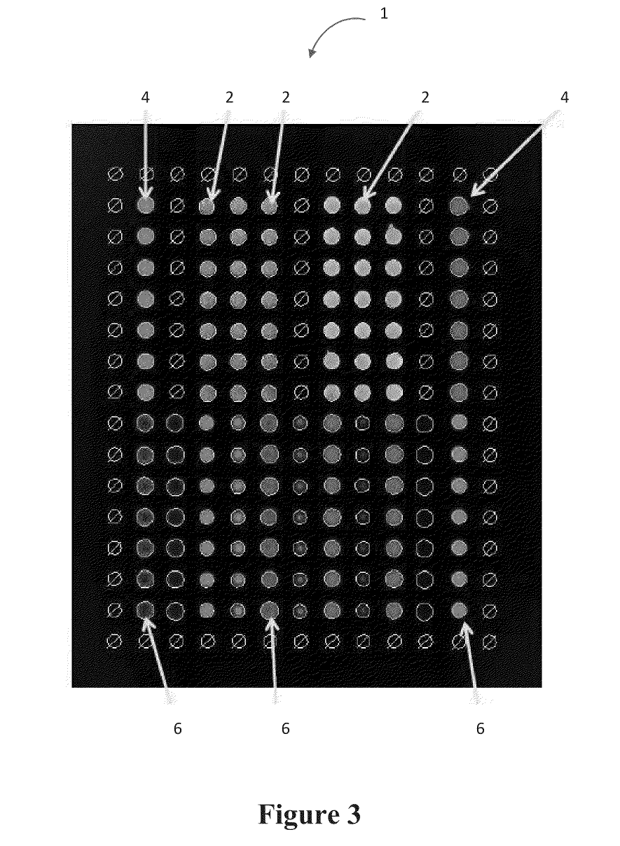

The microarray may comprise a calibration matrix comprising a series of calibration spots for each target analyte and an analyte capture matrix comprising one or more of test spots or capture spots which bind the target analytes. A representative microarray is shown in FIG. 3. The microarray 1 includes capture spots 2 and calibration spots 4. In addition, internal control spots 6 are included to ensure that the microarray is functioning properly.

As used herein, the term “calibration matrix” refers to a subarray of spots printed on and adhering to the reaction vessel, wherein each spot comprises a predetermined amount of a calibration standard. The term “predetermined amount” as used herein, refers to the amount of the calibration standard as calculated based on the known concentration of the spotting buffer comprising the calibration standard and the known volume of the spotting buffer printed on the reaction vessel.

The choice of the calibration standard will depend on the nature of the target analyte. In such embodiments, the microarray will comprise a separate calibration standard for each target analyte. Alternatively, the microarray may comprise a single calibration matrix having calibration spots containing each of the target analytes.

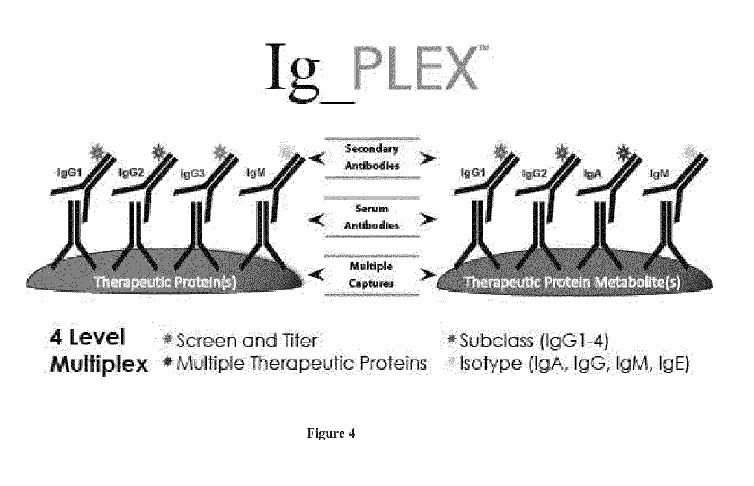

In alternate embodiments, the calibration standard is a surrogate compound. For example, if the target analyte is an antibody, the surrogate compound may be another, different antibody, but of the same class of immunoglobulin (FIG. 4). The calibration matrix may be printed on the base of the individual reaction vessel in the format of a linear, proportional dilution series. The predetermined concentrations of calibration standards are selected to include lower and upper expected detection limits to define the dynamic range. The mid-point of dynamic calibrated concentration range approximates the diagnostic critical concentration of the detection system used to read the microarray.

A person skilled in the art will appreciate that the method of the present invention can be carried out without the use of calibration dots or matrices. Measurements of the intensity of signal from the capture dots can be calculated with reference to known external standards. As such, the determination of the amount of analytes is made without using internal dynamic calibration in alternate embodiments of the method of the present invention.

As used herein, the term “analyte capture matrix” refers to a subarray of spots comprising agents that selectively bind the target analytes. In embodiments where the target analyte is a protein, the agent may be an analyte specific antibody or fragment thereof. Conversely, in embodiments wherein the target analyte is an antibody, the agent may be an antigen specifically bound by the antibody. For example, FIG. 4 illustrates the capturing of five different antibodies that selectively bind to two different antigens.

A predetermined volume of a test sample is applied to the assay device. Each of the target analytes will bind to their specific capture spot. Thus, in a single capture spot, multiple target analytes may be bound. To detect each of the target analytes, a fluorescently labelled antibody that specifically binds to the target analyte is used. Each fluorescently labelled antibody is coupled to a unique fluorescent dye with a specific excitation and emission wavelength to obtain the desired Stokes shift and excitation and emission coefficients. The fluorescent dyes are chosen based on their respective excitation and emission spectra such that each of the labelled antibodies comprises a different fluorescent dye having emission and excitation spectra which do not overlap with each other. The fluorescently labelled antibodies can be applied to the assay device in a single step in the form of a cocktail.

A signal intensity value for each spot within the assay device is then measured as shown in FIG. 5. The fluorescent signals can be read using a combination of scanner components such as light sources and filters. A signal detector can be used to read one optical channel at a time such that each spot is imaged with multiple wavelengths, each wavelength being specific for a target analyte. An optical channel is a combination of an excitation source and an excitation filter, matched for the excitation at a specific wavelength. The emission filter and emission detector pass only a signal wavelength for a specific fluorescent dye. The optical channels used for a set of detectors are selected such that they do not interfere with each other, i.e. the excitation through one channel excites only the intended dye, not any other dyes. Alternatively, a multi-channel detector can be used to detect each of the differentially labelled antibodies. The use of differential fluorescent labels allows for substantially simultaneous detection of the multiple target analytes bound to a single capture spot.

The measured signal intensity is directly proportional to the amount of material contained within the printed calibration spots and the amount of analyte from the test sample bound to the printed analyte capture spot. For each calibration compound, a calibration curve is generated by fitting a curve to the measured signal intensity values versus the known concentration of the calibration compound. The concentration for each target analyte in the test sample is then determined using the appropriate calibration curve and by plotting the measured signal intensity for the target analyte on the calibration curve.

The method disclosed herein can be used to detect and quantify multiple clinically relevant biomarkers in a biological sample for diagnostic or prognostic purposes. The measured concentrations for a disease related biomarker can be compared with established index normal levels for that biomarker. The measured concentrations levels which exceed index normal levels may be identified as being diagnostic of the disease. The method disclosed herein can also be used to monitor the progress of a disease and also the effect of a treatment on the disease. Levels of a clinically relevant biomarker can be quantified using the disclosed method a plurality of times during a period of treatment. A trending decrease in biomarker levels may be correlated with a positive and/or negative patient response to treatment.

The method disclosed herein can be used to detect and quantify biomarkers diagnostic for insulin immunogenicity. In one embodiment, the method comprises the provision of an assay device having a microarray printed thereon. The microarray may comprise: i) a calibration matrix comprising plurality of calibration spots, each calibration spot comprising a predetermined amount of one of: a human IgA antibody, a human IgG antibody, a human IgM antibody, a human IgE antibody and respective subclasses; ii) a first analyte capture matrix comprising a plurality of capture spots comprising a predetermined amount of a compound, for example, insulin; and optionally iii) a second analyte capture matrix comprising a plurality of capture spots comprising a predetermined amount of anti-insulin peptide. A predetermined volume of a biological sample, preferably a serum sample, is applied to the assay device. A cocktail comprising a first fluorescently labelled reporter compound that selectively binds to IgA antibodies, a second fluorescently labelled reporter compound that selectively binds to IgG antibodies, a third fluorescently labelled reporter compound that selectively binds to IgM antibodies, a fourth fluorescently labelled reporter which selective binds to IgE and fluorescent labels which bind selectively to immunoglobulin subclasses, is then applied to the assay device. The first, second, third, fourth and selected subclass fluorescently labelled antibodies are chosen such that each of the antibodies comprises a different fluorescent dye having emission and excitation spectra which do not overlap with each other, as shown in FIG. 5. A signal intensity value for each spot within the assay device is then measured using a single or multi-channel detector as discussed above. Using the measured signal intensity values, calibration curves are then generated by fitting a curve to the measured signal intensity values for each of the calibration spots versus the concentration of the human IgA, IgG, IgM, IgE and subclass antibodies. The concentration for each of the captured insulin analytes is determined using the calibration curves.

In certain embodiments, the method disclosed herein may be used, e.g., to diagnose or monitor the progress of autoimmune diseases. In other embodiments, the method disclosed herein can be used for monitoring the progress of treatment.

### Example 1

### Multiplex Immunogenicity Testing

Wherein a therapeutic protein and/or its analytical components are immobilized on a planar microarray surface, analytical components may include subunits of the therapeutic protein e.g. antibody fragments or fusion partners, metabolic products of the therapeutic protein, peptide components, formulation components, biosimilars, or potential cross reacting entities.

Samples collected from untreated and therapeutic protein treated patients are incubated with the immobilized microarray components. Samples are most likely to be serum or plasma. These samples may be pre-treated or prepared in such a way as to enrich for the availability of any antibodies which the patient may have developed in response to the therapeutic protein or prior exposure to similar entities. Following sample incubation, the microarray surface is interrogated for the presence of patient derived antibodies which have been captured and bound by the immobilized analytes.

The amount of and heavy chain characteristics of the captured patient antibodies are determined by the use of specific anti-human secondary antibodies which have been conjugated to fluorescent dyes.

Secondary reagents can be included to determine the immunoglobulin class Ig G, A, M or E or the sub-classes, including IgG1, IgG2, IgG3, and IgG4. Specific dyes are conjugated to each of the secondary reagents to constitute a reporter and allow differentiation of each of the Ig classes or subclasses. A reporter aliquot is made up of a mix of conjugates as determined by the classes and subclasses that are of interest in the patient study.

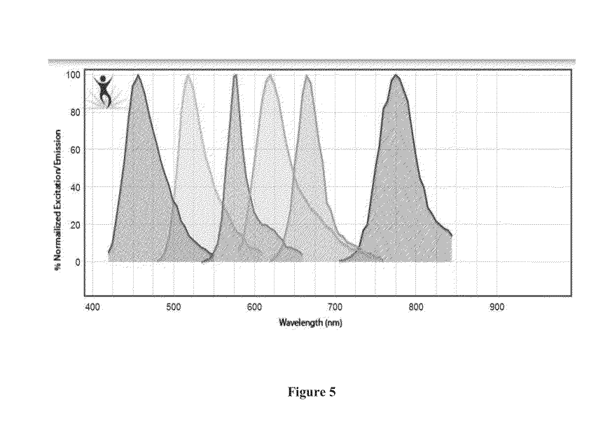

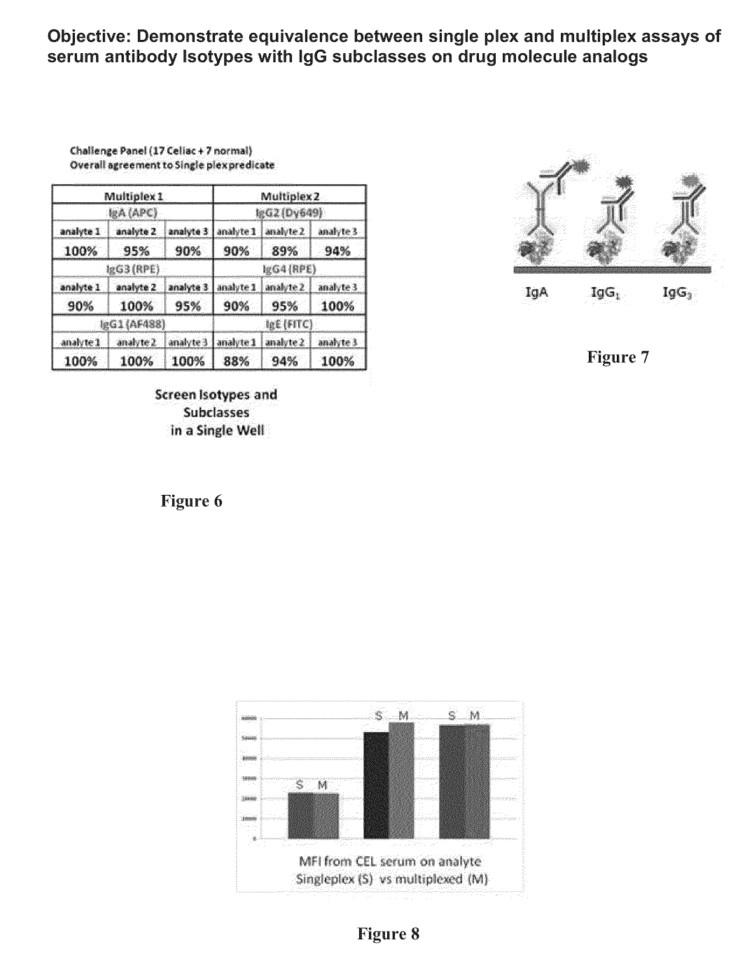

As shown in FIGS. 5, 6 and 7, this assay can be configured to use (i) a three color fluorescent scanner by including the same patient sample in multiple interrogated wells and adding a three color constituent reporter blend to each of the wells; or (ii) increased to multiples of up to six colors per well as determined by selecting fluorescent dyes which have separable emission peaks used in conjunction with a scanner equipped with appropriate excitation and emission filters.

The intensity of the multiple fluorescent signals when compared to standard curves intensities will allow the qualitative and quantified measurement of a specific immune response to the therapeutic protein or the protein associated analytes.

As one demonstration of the utility of this method, the sensitivity and response of the multiplexed assay for each of the subclasses can be shown to be equivalent to the single plex (one isotype measured at a time) performance. FIGS. 5 and 7 show the results.

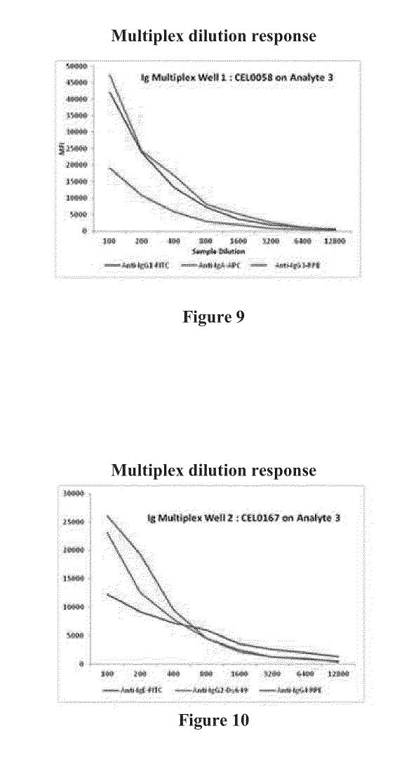

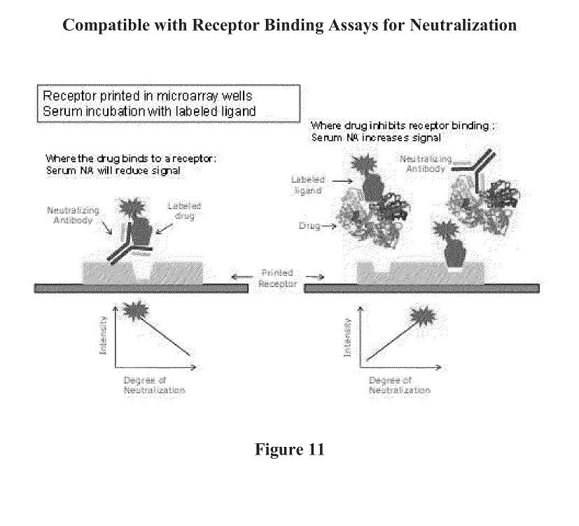

The signal intensity from multiple isotypes decreases as a sample undergoes serial dilution to develop a coordinated standard curve for quantitation or semi-quantitation of a multiplexed assay, as shown in FIGS. 8 and 9.

### Example 2

### Neutralizing Antibodies

The method also interrogates neutralizing effects of a patient's antibodies, i.e.; their ability to directly affect the active mechanism of the therapeutic protein

In cases where the therapeutic protein is a ligand that binds to a receptor, the receptor will be immobilized on the array surface. A fluorescently labeled derivative of the therapeutic protein will be incubated with patient serum in a competitive type immunoassay. A high fluorescent signal in this case indicates an absence of neutralizing antibodies. As the titer of neutralizing antibodies increases in a sample, they will interfere with the ability of the labeled therapeutic protein to bind the receptor and thus decrease the florescent signal on the array surface.

As depicted in FIG. 10, in cases where the mechanism of the therapeutic protein is to block a ligand/receptor interaction, the receptor is immobilized on the microarray surface. A fluorescently labeled derivative of the appropriate ligand, and the therapeutic protein is incubated with patient serum. In the absence of neutralizing antibodies the therapeutic protein will block the binding of the labeled ligand to the receptor and little or no fluorescent signal will be detected. As the titer of neutralizing antibodies increases, they will interfere with the ability of the therapeutic protein to block the ligand/receptor interaction, and the fluorescent signal will increase.

### Example 3

### Insulin Immunogenicity

As new insulin variants are developed the need to study the range of immune responses in patients requires the ability to detect, characterize and quantitate anti-insulin antibodies. Regardless of purity and origin, therapeutic insulins continue to be immunogenic in humans. Severe immunological complications rarely occur. Current human insulin and insulin analog therapies result in decreased anti-insulin antibodies levels. Anti-insulin antibody development is also affected by the mode of delivery: For example, use of subcutaneous and implantable insulin pumps or inhaled insulin. Formulation also effects immunogenic potential with regular or semilente insulins being less immunogenic than intermediate or long acting preparations. Aggregation levels also affect immunogenicity.

Anti-insulin antibodies responses consisting of Ig classes and IgG subclasses have been reported. Primarily IgG1-4 but IgA, IgM and IgE have also been reported. IgG is implicated in the most severe cases of insulin resistance. Insulin delivered or inhaled results in a similar distribution of IgG subclasses: IgG1>IgG4>IgG2 and IgG3. IgG1 levels have been reported to decline where IgG4 rises with increased duration of insulin treatment.

The method disclosed is uniquely suited to detect and differentiate the range of anti-insulin antibodies in a single assay, as opposed to running a separate assay for each Ig class or subclass.

In this case, the therapeutic insulin is printed as multiple replicate spots in each well of a 96 well functionalized glass plate. The print conditions, including buffers, concentration, and post print processes are selected to optimize epitope presentation and assay precision. Assay controls including anti-human antibodies or other variants of insulin could be included in each of the 96 wells.

Each well is incubated with patient serum. In cases where the patient has anti-insulin antibodies they are captured by the spotted insulin. Fluorescently labeled anti-human Ig secondary reagents are used to detect the binding anti-insulin antibodies. Secondary reagents include Ig Class specific (IgG, IgA, IgM or IgE) or subclass specific (IgG1, IgG2, IgG3, IgG4). A fluorescent dye with a different emission spectrum is conjugated to each of the secondary reagents allowing the patient immune response to be characterized based on the intensity of each signal.

In the case where a commercial 3-color array scanner is used to detect the fluorescent signals, the same patient sample is interrogated in multiple wells and different aliquots of labeled reporters are used to fluorescently label each reporter in each well with a specific dye wavelength, e.g. in Well 1, IgA is labeled with dye Cy5 at 667 nm (nanometers), IgG1 with dye FITC at 488 nm and IgG3 with dye PE at 575 nm to measure the IgA, IgG1 and IgG3 in Well 1. In Well 2, IgM is labeled with Cy5 at 667 nm, IgG2 is labeled with FITC at 488 nm and IgG4 is labeled with PE to fluoresce at 575 nm to measure IgM, IgG2 and IgG4. The six immunoglobulins are measured using only three fluorescing labels.

For simultaneous detection of up to six different color fluorescent wavelengths per well, as illustrated in FIG. 5, the excitation source and emission filters are coordinated with a set of dyes with compatible spectra; IGg1—FITC dye at 488 nm, IgG2—PE dye at 575 nm, IgG3—Cy5 dye at 667 nm, IgG4—Pe-Cy dye at 767 nm, IgA—Pac Blue dye at 457 nm, IgM—Alexa fluor dye at 594 615 nm. If more than six fluorescent labels are required to report and measure multiple antigens, the identified set, or compatible sets of six wavelengths are applied in each separate well. It is to be understood that more than six different color fluorescent wavelengths per well can be detected in alternate embodiments hereof.

### Example 4

### Anti Drug Antibody (ADA) Specificity Confirmation

A method to demonstrate antibody specificity is using a competition assay where increasing amounts of the free drug are added to the sample as the assay signal decreases. In addition to being able to quantitate and isotype ADAs this method can also be applied to simultaneously demonstrate the specificity of each of the isotypes detected.

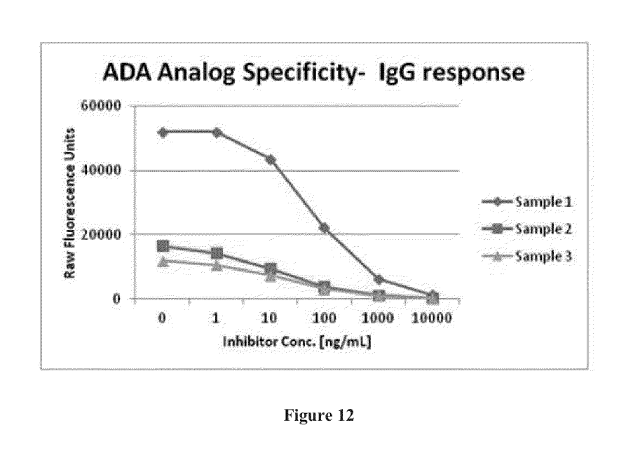

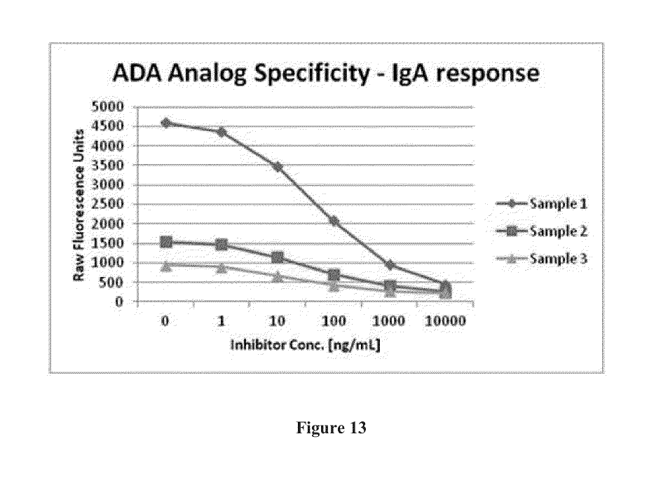

By adding free drug to the serum all of the isotypes that are specific to the drug will show a decrease in signal. This specificity demonstration can either be carried out at the same time with sample +/− drug being added to 2 different wells of the assay, or as a follow on assay to confirm specificity in samples showing positive signal. Results are shown in two graphs namely FIGS. 12 and 13.

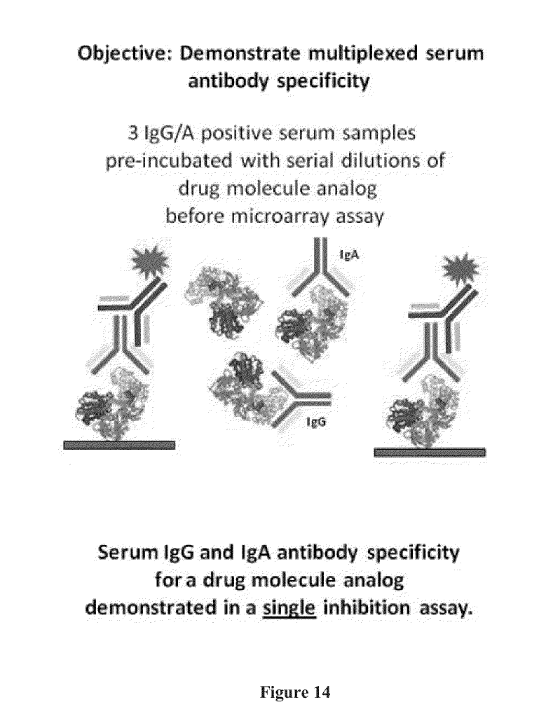

FIG. 14 is an illustration showing as the objective of this example to demonstrate multiplexed serum antibody specificity. In the example, 3 IgG/A positive serum samples pre-incubated with serial dilutions of drug molecule analog before microarray assay are depicted. Serum IgG and IgA antibody specificity for a drug molecule analog is demonstrated in a single inhibition assay.

### Example 5

### Multiplexed Compatibility with Acid Dissociation

Drug Tolerance is defined as the maximum amount of free drug that can be present in a sample and still allow detection of ADAs. The presence of high level of free drug may cause anti-drug antibodies to be sequestered in immuno-complexes and unavailable to bind the capture analyte in an immunoassay. Acid dissociation disrupts the immune complexes and improves the drug tolerance of the assay. The ability to form the complexes and also to have binding disrupted by acid or other chemical treatment is independent of isotype. For this application the serum is pre-treated with a disruptive agent. The acid is neutralized before adding the sample to the well. The multiplexed reporter cocktail is used to detect and quantitate each of the isotype or subclasses involved in the anti-drug response.

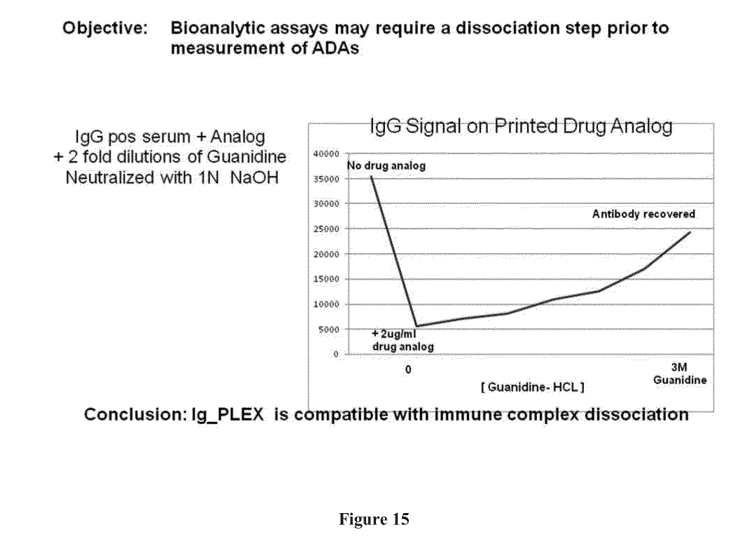

FIG. 15 graphically depicts an example of this assay showing that bioanalytic assays may require a dissociation step prior to measurement of anti-drug antibodies. IgG positive serum plus analog plus two-fold dilutions of guanidine are neutralized with 1N NaOH. The graph shows IgG signal on printed drug analog. In conclusion the multiplex assay is compatible with immune complex dissociation.

Various embodiments hereof having been thus described in detail by way of example, it will be apparent to those skilled in the art that variations and modifications may be made without departing from the invention. Also included are all such variations and modifications as fall within the scope of the appended claims.

What is claimed is:

1. A method for simultaneously detecting and quantifying two or more target analytes in a test sample comprising two or more target analytes, contained in a single reaction vessel, the method comprising:
a) providing a reaction vessel having a microarray printed thereon, the microarray comprising:
i) a first calibration matrix comprising a plurality of first calibration spots, each of the first calibration spots comprising a predetermined amount of a first target analyte, the first target analyte being an antibody isotype or an antibody sub-class,
ii) a second calibration matrix comprising a plurality of second calibration spots, each of the second calibration spots comprising a predetermined amount of a second target analyte, the second target analyte being an antibody isotype or an antibody sub-class
iii) a first capture matrix comprising a plurality of first capture spots, each of the first capture spots comprising a predetermined amount of a first agent that selectively binds to the target analytes, and
iv) a second capture matrix comprising a plurality of second capture spots, each of the second capture spots comprising a predetermined amount of a second agent that selectively binds to the target analytes, and
b) adding a predetermined volume of the test sample to the microarray;
c) simultaneously applying at least two fluorescently labelled antibodies into the same well, each of the at least two fluorescently labelled antibodies being specific for one of the target analytes for selectively binding to one of the target analytes for individual identification and quantification of the target analytes, each of the fluorescently labelled antibodies comprising a different fluorescent dye having emission and excitation spectra which do not overlap with each other;
d) measuring signal intensity values for each fluorescently wavelength for each calibration spot and each capture spot within the microarray;
e) generating calibration curves by fitting a curve to the measured signal intensity values for each of the calibration spots versus a known concentration of the first target analyte and the second target analyte in the calibration spots; and
f) determining the concentration for the first target analyte and the second target analytes bound to the capture spots using the generated calibration curves.

2. The method according to claim 1, wherein a plurality of analytes are simultaneously detected.

3. The method according to claim 2, wherein the reaction vessel is a well of a multi-well plate and wherein each well has the microarray printed therein.

4. The method according to claim 1, wherein the test sample is a biological sample.

5. A method for detecting and quantifying immunoglobulins and/or biomarkers diagnostic for insulin immunogenicity, the method comprising:
a) providing an assay device having a microarray printed thereon, the microarray comprising:
i) a calibration matrix comprising plurality of calibration spots, each calibration spot comprising a predetermined amount of a single immunoglobulin class selected from the group consisting of IgA, IgG, IgM, IgD and IgE or a subclass thereof; and
ii) an analyte capture matrix comprising a plurality of capture spots, each capture spot comprising a predetermined amount of an agent that selectively binds to an immunoglobulin class selected from the group consisting of IgA, IgG, IgM, IgD and IgE or a subclass thereof;
b) applying a predetermined volume of a serum sample to the assay device;
c) applying a first fluorescently labelled antibody that selectively binds to IgA antibodies, a second fluorescently labelled antibody that selectively binds to IgG antibodies, a third fluorescently labelled antibody that selectively binds to IgM antibodies, a fourth fluorescently labelled antibody that selectively binds to IgE antibodies, a fifth fluorescently labelled antibody that selectively binds to IgE antibodies and a sixth fluorescently labelled antibody that selectively binds to respective sub-class antibodies to the assay device, wherein the first, second, third, fourth, fifth and sixth fluorescently labelled antibodies each comprise a different fluorescent dye having emission and excitation spectra which do not overlap with each other;
d) measuring signal intensity values for each fluorescently wavelength for each calibration spot and each capture spot within the microarray;
e) generating calibration curves by fitting a curve to the measured signal intensity values for the each of the calibration spots versus the known concentration of the human IgA, IgG, IgM, IgE, IgD and subclass immunoglobulins; and
f) determining the concentration for each captured analyte using the calibration curves.

6. A method for diagnosing neutralizing antibodies neutralizing therapeutic protein insulin in a subject, the method comprising:
a) providing an assay device having a microarray printed thereon, the microarray comprising:
iii) a calibration matrix comprising plurality of calibration spots, each calibration spot comprising a predetermined amount of a single immunoglobulin class selected from the group consisting of neutralizing factor-IgA, neutralizing factor-IgG, neutralizing factor-IgM, neutralizing factor-IgE, anti-insulin peptide-IgG, anti-insulin peptide-IgA, anti-insulin peptide-IgM and anti-insulin peptide-IgE;
iv) an analyte capture matrix comprising a plurality of capture spots, each capture spot comprising a predetermined amount of an agent that selectively binds to an immunoglobulin class selected from the group consisting of neutralizing factor-IgA, neutralizing factor-IgG, neutralizing factor-IgM, neutralizing factor-IgE, anti-insulin peptide-IgG, anti-insulin peptide-IgA, anti-insulin peptide-IgM and anti-insulin peptide-IgE;
b) applying a predetermined volume of a serum sample to the assay device;
c) applying a first fluorescently labelled antibody that selectively binds to neutralizing factor IgA antibodies, a second fluorescently labelled antibody that selectively binds to neutralizing factor IgG antibodies, a third fluorescently labelled antibody that selectively binds to IgM antibodies, a fourth fluorescently labelled antibody that selectively binds to neutralizing factor IgE antibodies, a fourth fluorescently labelled antibody that selectively binds to neutralizing factor IgM antibodies, a fifth fluorescently labelled antibody that selectively binds to anti-insulin peptide-IgG, a sixth fluorescently labelled antibody that selectively binds to anti-insulin peptide-IgA, a seventh fluorescently labelled antibody that selectively binds to anti-insulin peptide-IgM and an eighth fluorescently labelled antibody that selectively binds to anti-insulin peptide-IgE to the assay device, wherein the first, second, third, fourth, fifth, sixth, seventh and eighth fluorescently labelled antibodies each comprise a different fluorescent dye having emission and excitation spectra which do not overlap with each other;
d) measuring signal intensity values for each fluorescently wavelength for each calibration spot and each capture spot within the microarray;
e) generating calibration curves by fitting a curve to the measured signal intensity values for the each of the calibration spots versus the known concentration of neutralizing factor-IgA, neutralizing factor-IgG, neutralizing factor-IgM, neutralizing factor-IgE, anti-insulin peptide-IgG, anti-insulin peptide-IgA, anti-insulin peptide-IgM and anti-insulin peptide-IgE; and
f) determining the concentration levels of neutralizing factor-IgA, neutralizing factor-IgG, neutralizing factor-IgM, neutralizing factor-IgE and at least one of anti-insulin peptide-IgG, anti-insulin peptide-IgA, anti-insulin peptide-IgM and anti-insulin peptide-IgE in a biological sample, using the calibration curves.

7. A method for simultaneously detecting and quantifying two or more different target analytes in a test sample, the method comprising:
(a) providing a reaction vessel having a microarray printed thereon, the microarray comprising:
i) a calibration matrix comprising, for each of the target analytes, a plurality of calibration spots, each calibration spot comprising a predetermined known amount of a the target analyte in question,
iii) a capture matrix for each of the target analytes, comprising a plurality of capture spots, each capture spot comprising a predetermined amount of a binding agent that selectively binds to the target analytes in question, and
b) applying to the microarray a predetermined volume of the test sample to the microarray;
c) applying to the microarray a fluorescently labelled antibody specific to each of the target analytes, wherein each fluorescently labelled antibody selectively binds to the target analyte in question, and wherein each of the fluorescently labelled antibodies comprises a different fluorescent dye having emission and excitation spectra which do not overlap with each other;
d) measuring signal intensity values for each spot within the microarray;
e) generating a calibration curve for each target analyte by fitting a curve to the measured signal intensity values of each of the calibration spots for the target analyte in question versus the known concentrations of the calibration spots for the target analyte; and
f) determining the concentration for each target analyte using the corresponding calibration curves for the target analyte.

8. The method according to claim 7, wherein the target analytes are proteins.

9. The method according to claim 8, wherein the proteins are antibodies.

10. A method according to claim 7 wherein the step of measuring a signal intensity value for each spot within the microarray is carried out with a signal detector that is used to read one optical channel at a time.

11. A method for detecting and quantifying biomarkers diagnostic for rheumatoid arthritis, wherein the biomarkers comprise two or more target analytes in a serum sample, the method comprising:
a) providing an assay device having a microarray printed thereon, the microarray comprising:
i) a calibration matrix comprising, for each target analyte, a plurality of spots for each target analyte, each spot comprising a predetermined amount of the target analyte, and wherein the target analytes are a human IgA antibody, a human IgG antibody, and a human IgM antibody;
ii) a first analyte capture matrix comprising a plurality of spots, each spot comprising a predetermined amount of rheumatoid factor; and
iii) a second analyte capture matrix comprising a plurality of spots, each spot comprising a predetermined amount of cyclic citrullinated peptide;
b) applying a predetermined volume of the serum sample to the assay device;
c) applying a first fluorescently labelled antibody that selectively binds to IgA antibodies, a second fluorescently labelled antibody that selectively binds to IgG antibodies, and a third fluorescently labelled antibody that selectively binds to IgM antibodies to the assay device, wherein the first, second and third fluorescently labelled antibodies each comprise a different fluorescent dye having emission and excitation spectra which do not overlap with each other;
d) measuring signal intensity values for each spot within the assay device;
e) generating a calibration curve for each of the IgA, IgG and IgM antibodies, by fitting a curve to the measured signal intensity values of the calibration spots for the antibody in question versus the known concentration of the antibody; and
f) determining the concentration for each of captured rheumatoid factor-IgA, rheumatoid factor-IgG, rheumatoid factor-IgM, anti-cyclic citrullinated peptide-IgG, anti-cyclic citrullinated peptide-IgA, and/or anti-cyclic citrullinated peptide-IgM, in the first and second capture matrices, using the calibration curves for each of the IgA, IgG and IgM antibodies;
wherein the detection and quantification of rheumatoid factor antibodies, and
anti-cyclic citrullinated peptide antibodies is diagnostic for a stage of rheumatoid arthritis.

12. The method according to claim 11, further comprising:
diagnosing rheumatoid arthritis in a subject, comprising comparing the measured concentration levels of rheumatoid factor-IgA, rheumatoid factor-IgG, rheumatoid factor-IgM, anti-cyclic citrullinated peptide-IgG, anti-cyclic citrullinated peptide-IgA, and/or anti-cyclic citrullinated peptide-IgM with index normal levels of rheumatoid factor-IgA, rheumatoid factor-IgG, rheumatoid factor-IgM and anti-cyclic citrullinated peptide-IgG, anti-cyclic citrullinated peptide-IgA, and/or anti-cyclic citrullinated peptide-IgM wherein measured concentrations levels which exceed index normal levels is diagnostic for rheumatoid arthritis.

13. The method of claim 12, wherein detection and quantification of predominantly rheumatoid factor-IgM and anti-cyclic citrullinated peptide-IgM antibodies is diagnostic for an early stage of rheumatoid arthritis.

14. The method of claim 12, wherein the detection and quantification of rheumatoid factor-IgA and anti-cyclic citrullinated peptide-IgA antibodies is diagnostic for a transitional stage of rheumatoid arthritis.

15. The method of claim 12, wherein the detection and quantification of rheumatoid factor-IgG and anti-cyclic citrullinated peptide-IgG antibodies is diagnostic for a late stage of rheumatoid arthritis.

16. The method according to claim 11, wherein the step of measuring a signal intensity value for each spot within the microarray is carried out with a signal detector that is used to read one optical channel at a time.

17. A method for simultaneously detecting and quantifying two or more different target analytes in a test sample, the method comprising:
(a) providing a reaction vessel having a microarray printed thereon, the microarray comprising: a capture matrix for each of the target analytes, comprising a plurality of capture spots, each capture spot comprising a predetermined amount of a binding agent that selectively binds to the target analytes in question, and
b) applying to the microarray a predetermined volume of the test sample to the microarray;
c) applying to the microarray a fluorescently labelled antibody specific to each of the target analytes, wherein each fluorescently labelled antibody selectively binds to the target analyte in question, and wherein each of the fluorescently labelled antibodies comprises a different fluorescent dye having emission and excitation spectra which do not overlap with each other;
d) measuring signal intensity values for each spot within the microarray;
e) determining the concentration for each target analyte with reference to an external standard.

18. The method according to claim 17 wherein the method is simultaneously carried out in multiple individual wells of a multiplex assay device, wherein each individual well detects multiple different antibody sub-classes and isotypes.
